(19) United States
(12) Patent Application Publication
(10) Pub. No.: US 2006/0153897 A1
(43) Pub. Date: Jul. 13, 2006

(54) Zinc-containing foods

(76) Inventors: Yoshitane Kojima, Osaka (JP); Hiromu Sakurai, Osaka (JP); Yutaka Yoshikawa, Osaka (JP)

Correspondence Address: EDWARDS & ANGELL, LLP, P.O. BOX 55874, BOSTON, MA, 02205, US

(21) Appl. No.: 11/371,971

(22) Filed: Mar. 8, 2006

Related U.S. Application Data
(63) Continuation of application No. 10/476,394, filed on Oct. 30, 2003.

(30) Foreign Application Priority Data
May 9, 2001 (JP) 2001-139284

(51) Int. Cl. A61K 47/00; A61K 33/32; A61K 38/05; A61K 38/04

(52) U.S. Cl. 424/439; 424/641; 514/6

(57) ABSTRACT

It is intended to provide foods such as health (supplementary) foods, nutrition (supplementary) foods and foods with health claims (foods for specified health uses and foods with nutrient function claims) containing zinc compositions which are less toxic and have a high insulin-like activity. Foods containing organic compounds capable of forming a complex with zinc and zinc sources. More specifically, foods such as health foods, health supplementary foods, nutrition foods nutrition supplementary foods and foods with health claims (foods for specified health uses and foods with nutrient function claims) containing organic compounds such as picolinic acid, L-amino acids, oligopeptides, niacin, maltol, etc. and organic zinc complexes or mineral acid salts of zinc containing these compounds as a ligand and having an effect of lowering blood glucose level.

### TECHNICAL FIELD

The present invention relates to foods containing zinc as an effective ingredient and having a hypoglycemic action such as health food, health supplementary food, nutrition food, nutrition supplementary food and health functional food (specific food for health and nutrition functional food).

### BACKGROUND ART

It is the current status that there is no other treating method for type 1 (insulin-dependent) diabetes than subcutaneous-injection of insulin and there has been a demand for the development of preventive agent, treating agent, health supplementary food and foods with health claims (foods for specified health uses and foods with nutrient function claims) which are able to be orally administered in place of insulin. With regard to type 2 (noninsulin-dependent) diabetes caused by stress, obesity, lack of exercise, aging, etc., various treating agents have been developed and used clinically, however none of them are panacea and often cause problems of side effects. For such purpose, vanadyl sulfate and bispicolinic acid/vanadyl complex have been commercially available in the United States as a health supplementary food.

On the other hand, zinc (II) ion which is known to be less toxic than vanadium has been known to have an insulin-like activity since around 1980 [L. Coulston and P. Dandona, “Insulin-like effects of Zn²⁺ on adipocytes”, Diabetes, 29, 665-7 (1980); J. M. May and C. S. Contoreggi, “The mechanism of the insulin-like effects of ionic zinc”, J. Biol. Chem., 257, 4362-8 (1982); A. Shisheva, D. Gefel and Y. Shechter, “Insulinlike effects of zinc ion in vitro and in vivo” (Zn²⁺ is the first agent other than vanadate that on oral administration is able to restore tissue ability to metabolism glucose), Diabetes, 41, 982-8 (1992); etc.]

On the other hand, zinc contained in beer yeast and seaweed extract has been commercially available also in Japan as a health supplementary food. With regard to zinc complexes, there have been Japanese patent application 2000-145849 (International application PCT/JP00/07991) “Hypoglycemic agent comprising a zinc (II) organic complex” and Japanese patent application 2001-24532 “Hypoglycemic agent” filed by the present inventors and three articles [Y. Yoshikawa, E. Ueda, K. Kawabe, H. Miyake, H. Sakurai and Y. Kojima, “New insulin-mimetic zinc (II) complexes; Bis-maltolato zinc (II) and bis-2-hydroxypyridine-N-oxide zinc (II) with Zn(O₄) coordination mode”, Chem. Lett., 2000, 874; Y. Yoshikawa, E. Ueda, H. Miyake, H. Sakurai and Y. Kojima, “Development of zinc (II) complexes with blood glucose normalizing effect”, Biomed. Res. Trace Elements, 11, 349-350 (2000); and Y. Yoshikawa, E. Ueda, H. Miyake, H. Sakurai and Y. Kojima, “Insulinomimetic bis(maltolato)zinc (II) complex: Blood glucose normalizing effect in KK-A^{y} mice with type 2 diabetes mellitus”, Biochem. Biophys. Res. Commun., 281, 1190-1193 (2001)].

### DISCLOSURE OF THE INVENTION

The present invention aims to provide foods such as health (supplementary) food, nutrition (supplementary) food and foods with health claims (foods for specified health uses and foods with nutrient function claims) containing a zinc composition which has low toxicity and high insulin-like activity.

### BRIEF DESCRIPTION OF THE DRAWINGS

FIG. 1 shows body weight change (upper graph) and blood glucose level change (lower graph) in KK-A^{y} mice (▴) administered with a feed containing maltol and a bis(maltol)/zinc complex and in KK-A^{y} mice (X) administered with a feed (control) containing no zinc source or the like.

FIG. 2 shows body weight change (upper graph) and blood glucose level change (lower graph) in KK-A^{y} mice (●) administered with a feed containing L-threonine and a bis(L-threonine)/zinc complex and in KK-A^{y} mice (X) administered with a feed (control) containing no zinc source or the like.

FIG. 3 shows body weight change (upper graph) and blood glucose level change (lower graph) in KK-A^{y} mice (▪) administered with a feed containing 6-methylpicolinic acid and a bis(6-methylpicolinic acid)/zinc complex and in KK-A^{y} mice (x) administered with a feed (control) containing no zinc source or the like.

### BEST MODE FOR CARRYING OUT THE INVENTION

The present invention relates to foods containing an organic compound which is able to form a complex with zinc and a zinc source.

The organic compound which is able to form a complex with zinc used in the present invention includes picolinic acids, L-amino acids, oligopeptides, niacins, maltols, etc., but it is not limited to them.

Specific examples of the picolinic acids are one or more compound(s) selected from the group consisting of picolinic acid, 3- and 6-methylpicolinic acid, 3-, 4- and 6-halogeno-picolinic acid and an amide thereof.

Further, the above-mentioned picolinic acids are picolinic acid which is a metabolite of tryptophan in vivo and derivatives thereof.

Specific examples of the L-amino acids are one or more compound(s) selected from the group consisting of fundamental amino acids such as threonine, valine, leucine, isoleucine, glycine, proline, arginine, glutamine, glutamic acid, asparagine, aspartic acid, alanine, lysine, serine, methionine, phenylalanine, tyrosine, cysteine, tryptophan and histidine and an amide thereof.

With regard to the above-mentioned L-amino acids, mixtures thereof have been commercially available as nutrition supplementary food for sports, beauty and health.

Examples of the oligopeptides are one or more compound(s) selected from the group consisting of dipeptides or derivatives thereof and tripeptides or derivatives thereof.

Specific examples of the oligopeptides are aspartame (L-asparagyl-L-phenylalanine methyl ester) and glycyl-N,N′-ethylene-bridged-L-alanyl-L-alanine ethyl ester.

The above-mentioned aspartame has been already commercially available as a sweetener for those who need to reduce ingestion of sugar such as patients suffering from diabetes mellitus.

Specific examples of niacins are nicotinamide, nicotinic acid and a mixture thereof.

The above-mentioned niacins are nicotinic acid which is a metabolite of tryptophan in vivo, nicotinamide and derivatives thereof.

Specific examples of maltols are maltol, ethylmaltol and a mixture thereof.

The above-mentioned maltols are maltol which is a food additive and derivatives thereof.

With regard to the organic compound which is able to form a complex with zinc in accordance with the present invention, the one derived from natural substance is particularly preferable.

With regard to the zinc source used in the present invention, anything may be used so far as it is a zinc source appropriate for administration to humans and/or other animals; for example, mineral acid salt of zinc and organic complex of zinc are preferable.

Examples of mineral acid salt of zinc are zinc chloride, zinc sulfate and zinc nitrate. When mineral acid salt of zinc is used as a zinc source, it is preferable to use a basic aqueous solution, such as sodium hydroxide, lithium hydroxide and barium hydroxide, or a buffer, such as a citrate buffer and a phosphate buffer, at the same time as a pH regulator.

With regard to the zinc organic complex, preferred examples are zinc organic complex having a compound selected from the group consisting of picolinic acids, L-amino acids, oligopeptides, niacins and maltols as a ligand.

With regard to the specific examples of the compound used as a ligand, those which are same as the corresponding compound in the organic compound which is able to form a complex with zinc may be listed.

With regard to the zinc source, zinc-containing yeast, etc. such as beer yeast may be also usable.

The food concerning the present invention may further contain other foods, food-additives, vitamins and/or minerals.

With regard to those foods, food additives, vitamins, minerals, etc., anything may be used so far as they have been used commonly in the fields of pharmaceuticals and foods or will be probably used therein in future. However, since the food according to the present invention is inherently a food having a hypoglycemic action such as health food, health supplementary food, nutrition food, nutrition supplementary food and foods with health claims (foods for specified health uses and foods with nutrient function claims), the use in combination with foods, food additives, vitamins, minerals, etc. which inhibit such action is naturally unpreferable.

Shape of the food concerning the present invention may be any of the shapes of powder, granules, tablets, capsules, liquid, gel and others.

Regardless of the above-mentioned shape, all foods and beverages in which the organic compound capable of forming a complex with zinc and the zinc source in accordance with the present invention is contained in the already-known food (food and beverage) correspond to the food of the present invention.

Examples of such food and beverage are beverage such as refreshing beverage, nutrition beverage, fruit beverage and lactic acid beverage (including concentrated original liquid and/or adjusted powder for the preparation of such a beverage); frozen dessert such as ice cream and sherbet; processed cereal such as soba, udon, bread, mochi and gyoza wrapper skins; confectionery such as candy, chocolate, snack food, biscuit, cookie, cracker, jelly and jam; processed food of fish and livestock such as kamaboko, hampen, ham and sausage; milk product such as processed milk, cheese and butter; fat/oil and processed fat/oil food such as margarine, lard and mayonnaise; seasoning such as soybean sauce, sauce, miso, ponzu, bouillon of kelp and instant soup; various prepared foods; pickles; and nutrition and health supplementary foods in various forms; however they are not limited to them.

The food according to the present invention containing an organic compound capable of forming a complex with zinc and a zinc source is greatly expected as a health (supplementary) food and foods with health claims (foods for specified health uses and foods with nutrient function claims) which normalizes the blood glucose level of patients suffering from diabetes mellitus, etc. and has an effect of prevention and treatment of impaired glucose tolerance, diabetes mellitus (type II diabetes mellitus, etc.), insulin-resistant syndrome (such as insulin receptor abnormalities), polycystic ovary syndrome, hyperlipemia, atherosclerosis, cardiovascular diseases (such as angina pectoris and cardiac insufficiency), hyperglycemia, hypertension, angina pectoris, pulmonary hypertension, congestive heart failure, complication with diabetes mellitus (such as diabetic gangrene, diabetic arthropathy, diabetic glomerulosclerosis, diabetic skin failure, diabetic neuropathy, diabetic cataract and diabetic retinopathy) or dysgerusia.

The food concerning the present invention is also greatly expected as a nutrition (supplementary) food which activates insulin and has an action of normalization of blood glucose.

### EXAMPLES

The present invention will be more specifically illustrated by the following Examples and Comparative Examples, however the present invention is not limited by those Examples and Comparative Examples at all.

### Example 1

Maltol was used as an organic compound which is able to form a complex with zinc while a bis(maltol)/zinc complex was used as a zinc source and the feed for administration in a large amount (feeding amount) was prepared according to the following formulation.

| | | |
|---|---|---|
| Corn starch | 2,929.6 | g |
| Granulated sugar | 3,300 | g |
| Egg white powder | 2,000 | g |
| Corn oil | 500 | g |
| KC Flock (cellulose powder) | 300 | g |
| Okanol (α-starch) | 100 | g |
| Vitamin Mix | 100 | g |
| Mineral Mix | 700 | g |
| Maltol | 64.0 | g |
| Bis(maltol)/zinc complex | 6.40 | g |
| Total | 10 | kg |

Components of Vitamin Mix (per 1 g)

| | | |
|---|---|---|
| Vitamin A and D₃ | 2.4 | mg |
| (VA 500,000 IU; VD₃ 100,000 IU/g) | | |
| Vitamin E (50%) | 20.0 | mg |
| Vitamin K₃ | 0.3 | mg |
| Vitamin B₁ | 1.5 | mg |
| Vitamin B₂ | 1.56 | mg |
| Vitamin B₆ | 1.02 | mg |
| Biotin (2%) | 0.5 | mg |
| Calcium dl-pantothenate | 4.0 | mg |
| PABA | 10.15 | mg |
| Nicotinic acid | 10.15 | mg |
| Inositol | 15.0 | mg |
| Folic acid | 0.2 | mg |
| Choline chloride | 300.0 | mg |
| Vitamin B₁₂ (0.1%) | 5.2 | mg |
| Corn starch | 628.02 | mg |
| Total | 1 | g |

Components of Mineral Mix (per 7 g)

| | | |
|---|---|---|
| CaCO₃ | 1,355.4 | mg |
| KH₂PO₄ | 1,730.0 | mg |
| CaHPO₄.2H₂O | 1,500.0 | mg |
| CaSO₄.7H₂O | 800.0 | mg |
| NaCl | 600.0 | mg |
| FeC₆H₅O₇.5H₂O | 190.0 | mg |
| 5ZnO.2CO₂.4H₂O | 6.0 | mg |
| CuSO₄.5H₂O | 1.26 | mg |
| CoCl₄.6H₂O | 0.4 | mg |
| Ca(IO₃)₂ | 1.54 | mg |
| MnSO₄.4H₂O | 15.4 | mg |
| Corn starch | 800.0 | mg |
| Total | 7 | g |

The feed I prepared according to the above-mentioned formulation was continuously administered in large quantities (5 g/day) for a long period to KK-A^{y} mice (8 weeks old) which were model animals for type 2 diabetes.

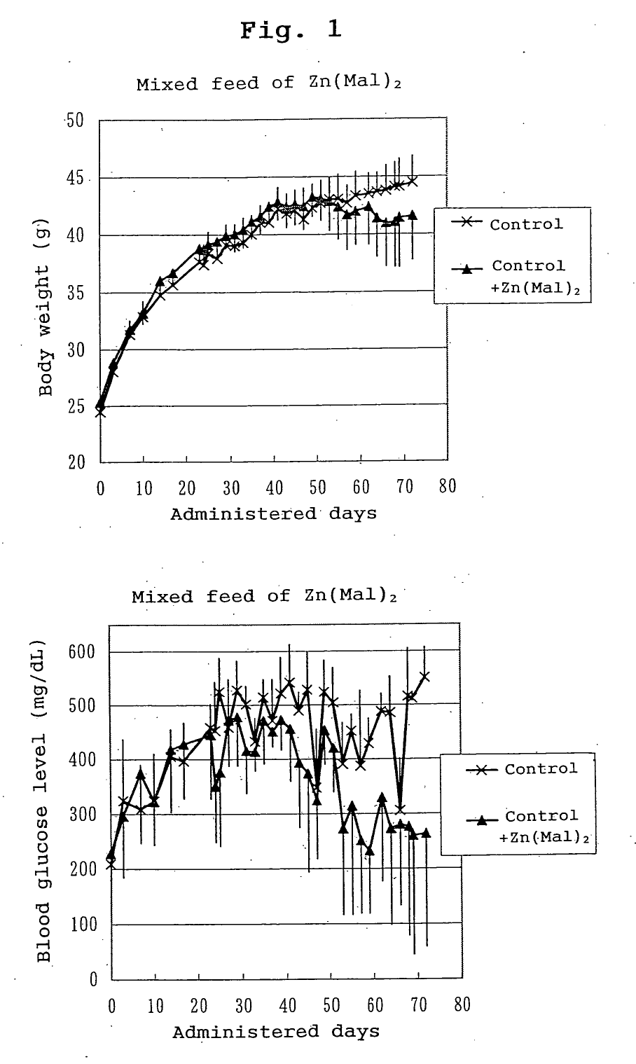

Changes in blood glucose level and body weight are shown in FIG. 1.

### Example 2

A feed II was prepared in the same manner as in the case of the feed I using L-threonine and a bis(L-threonine)/zinc complex instead of maltol and bis(maltol)/zinc complex in the feed I and was continuously administered in large quantities (5 g/day) for a long period to KK-A^{y} mice (8 weeks old) according to Example 1.

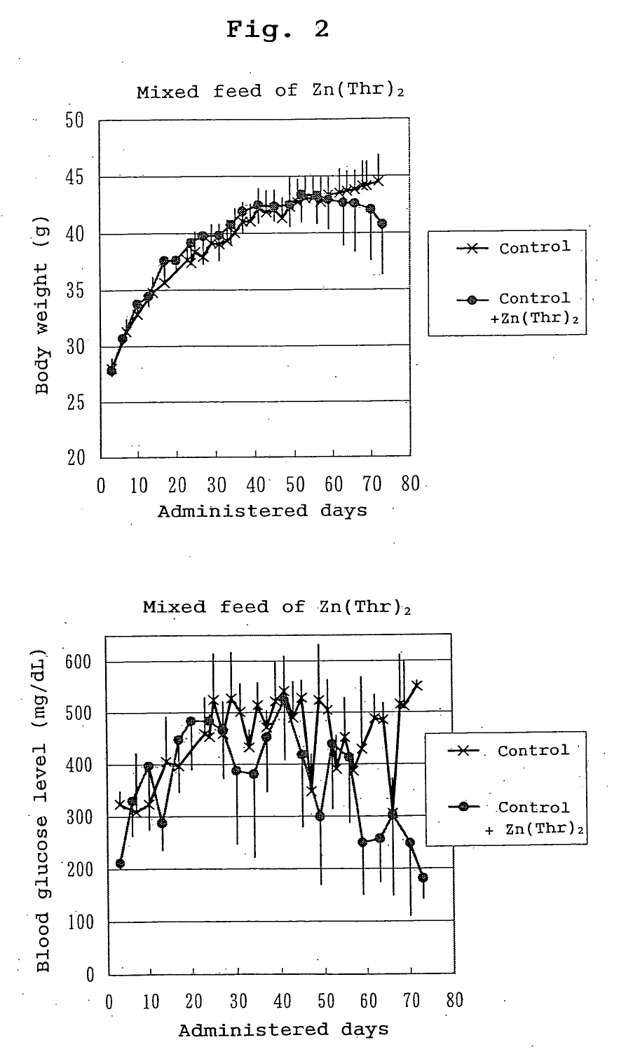

Changes in blood glucose level and body weight are shown in FIG. 2.

### Example 3

A feed III was prepared in the same manner as in the case of the feed I using 6-methylpicolinic acid and a bis(6-methylpicolinic acid)/zinc complex instead of maltol and bis(maltol)/zinc complex in the feed I and was continuously administered in large quantities (5 g/day) for a long period to KK-A^{y} mice (8 weeks old) according to Example 1.

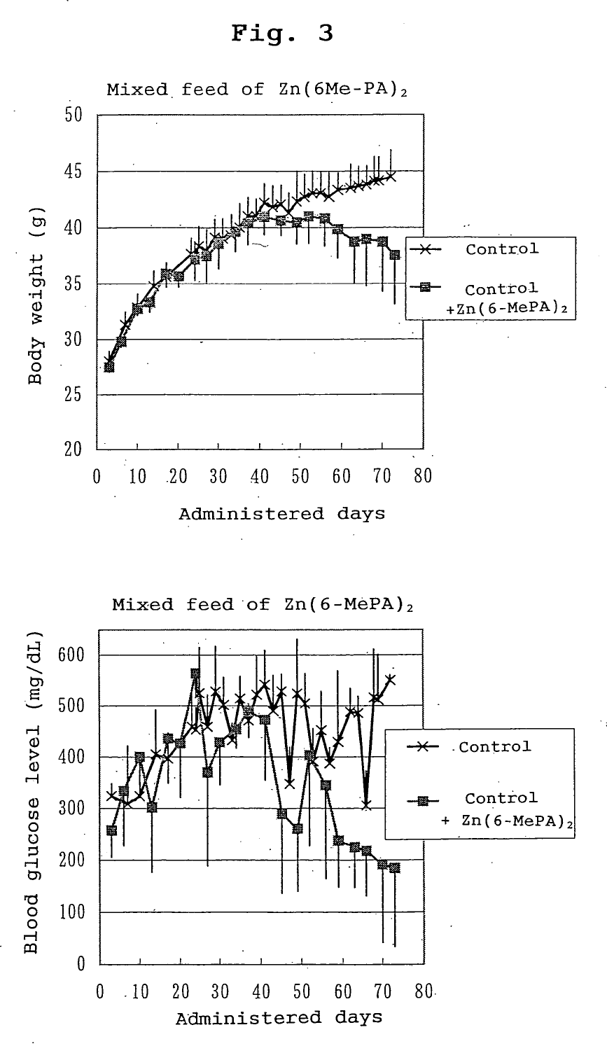

Changes in blood glucose level and body weight are shown in FIG. 3.

As being apparent from FIG. 1 to FIG. 3, when the food of the present invention containing an organic compound capable of forming a complex with zinc and a zinc source is administered, the blood glucose level clearly lowered on around the 40th to 50th day from the administration as compared with the control (where a feed containing no organic compound capable of forming a complex with zinc and a zinc source is administered).

Further, when the food of the present invention is administered, adjustment of body weight was occurred on around the 40th to 60th day after administration in spite of administration of large quantities.

In addition, with regard to the above-mentioned feed I, feed II and feed III, those which were formulated and prepared by Nippon Claire K. K. were used.

### Example 4

Water (30 g) was added to a mixture of 100 g of butter, 30 g of sugar, 200 g of flour, 0.5 to 5 g of baking soda, 20 g of L-amino acids mixture (each 4 g of valine, leucine, isoleucine, glutamine and arginine) and 2 g of a bis(L-threonine)/zinc complex followed by heating and baking to produce soft cookie.

### Example 5

Various kinds of soft cookies were produced by the same manner as in Example 4 except that, 0 to 50 g of L-amino acid mixture (a mixture of same amounts of valine, leucine, isoleucine, glutamine and arginine), 0.5 to 5 g of a bis(L-threonine)/zinc complex and 1 to 20 g of fish oil containing DHA were added in place of 20 g of L-amino acid mixture and 2 g of a bis(L-threonine)/zinc complex in Example 4.

### Example 6

To 1 to 50 g of L-amino acid mixture (a mixture of same amounts of valine, leucine, isoleucine, glutamine and arginine) and 0.2 to 5 g of zinc chloride were added a pH regulator (sodium hydroxide), a sweetener and powdered juice mixes to produce various kinds of juice.

Test Example 1. Acute Toxicity Test

Outside the application of GLP, acute toxicity test was carried out for zinc complexes with maltol, picolinic acid, picolinamide, 6-methyl-picolinic acid, L-threonine and L-valine.

With regard to zinc complexes with picolinic acid, 6-methyl-picolinic acid and L-valine, no special change was noted in the female and male rats after 2, 8 and 15 days from administration of 2,000 mg/kg in the normal state and autopsy compared with the females and males of a solvent control group, whereby it was concluded that the LD₅₀ values for those three complexes were not less than 2,000 mg/kg (limit dose for administration) for both females and males.

In the test carried out in the same manner, LD₅₀ values for maltol, L-threonine and picolinamide were 1676.1, 1108.0 and 1293.9 mg/kg, respectively for the case of males and were 1589.2, 1304.7 and 1212.5 mg/kg, respectively for the case of females.

Test Example 2. Mutagenicity Test

As a mutagenicity test (Ames) using microorganisms, mutation-inducing ability of zinc complexes with maltol, picolinic acid, picolinamide, 6-methyl-picolinic acid, L-threonine and L-valine was examined using Salmonella typhimurium TA 100, TA 1535, TA 98 and TA 1537 and Escherichia coli WP 2 uvr A.

Zinc complexes with picolinamide, 6-methyl-picolinic acid, L-threonine and L-valine were judged to have no mutation-inducing ability in all strains as compared with a solvent control.

Zinc complexes with maltol and picolinic acid showed growth inhibition at high concentrations in TA 1535 and TA 98, TA 1537 strains respectively, but under the test condition, it was judged to be very weakly positive. However, although maltol and picolinic acid per se showed growth inhibition at high concentrations, maltol was allowed as a food additive and is actually used as a food additive while picolinic acid is a metabolite of tryptophan in vivo whereby it was judged that there will be no problem even if they were administered in vivo.

### INDUSTRIAL APPLICABILITY

The present invention provides a food containing a zinc composition which has low toxicity and high insulin-like activity such as health (supplementary) food, nutrition (supplementary) food and foods with health claims (foods for specified health uses and foods with nutrient function claims) and, since the food concerning the present invention has a high insulin-like activity and a hypoglycemic action, it is greatly expected as health (supplementary) food, nutrition (supplementary) food and foods with health claims (foods for specified health uses and foods with nutrient function claims) having an effect of prevention and treatment of diseases such as impaired glucose tolerance, diabetes (such as type 2 diabetes) and insulin-resistant syndrome (such as insulin receptor abnormalities) in which insulin and blood glucose level are related.

1-11. (canceled)

12. A method for lowering the blood glucose level in a patient suffering from diabetes which comprises a) providing a composition containing a complex consisting of L-amino acids and zinc with the proviso that the complex does not have a hetero ligand, and b) administering the composition to the patient suffering from diabetes.

13. The method according to claim 12, wherein the L-amino acids are one or more compound(s) selected from the group consisting of threonine, valine, leucine, isoleucine, glycine, proline, arginine, glutamine, glutamic acid, asparagine, aspartic acid, alanine, lysine, serine, methionine, phenylalanine, tyrosine, cysteine, tryptophan, histidine and an amide thereof.

14. The method according to claims 12 or 13, wherein the composition further comprises foods, food additives, vitamins and/or minerals.
